(19) United States
(12) Patent Application Publication
(10) Pub. No.: US 2012/0087984 A1
(43) Pub. Date: Apr. 12, 2012

(54) STABILIZATION OF CHEMICAL COMPOUNDS USING NANOPARTICULATE FORMULATIONS

(75) Inventors: Elaine LIVERSIDGE, West Chester, PA (US); Linden Wei, Exton, PA (US)

(73) Assignee: Elan Pharma International Ltd.

(21) Appl. No.: 13/252,143

(22) Filed: Oct. 3, 2011

Related U.S. Application Data
(63) Continuation of application No. 11/979,251, filed on Oct. 31, 2007, now abandoned.
Application No. 11/979,251 is a continuation of application No. 09/952,032, filed on Sep. 14, 2001.

(51) Int. Cl. A61K 9/14; A61P 35/00; A61K 31/337; C07D 305/14; B82Y 40/00; B82Y 5/00

(52) U.S. Cl. 424/490; 514/449; 549/201; 428/403; 977/773; 977/915; 977/882

(57) ABSTRACT

Methods for stabilizing chemical compounds, particularly pharmaceutical agents, using nanoparticulate compositions are described. The nanoparticulate compositions comprise a chemical compound, such as a pharmaceutical agent, and at least one surface stabilizer. The component chemical compound exhibits chemical stability, even following prolonged storage, repeated freezing-thawing cycles, exposure to elevated temperatures, or exposure to non-physiological pH conditions.

### CROSS-REFERENCE TO RELATED APPLICATIONS

This application is a Continuation of U.S. patent application Ser. No. 11/979,251, filed Oct. 31, 2007, which is a Continuation of U.S. patent application Ser. No. 09/952,032, filed Sep. 14, 2001. The contents of these applications are incorporated herein by reference in their entirety.

### FIELD OF THE INVENTION

The present invention is directed to methods for stabilizing chemical compounds, particularly pharmaceutical agents, comprising formulating a chemical compound into a nanoparticulate composition. The nanoparticulate composition comprises a chemical compound and one or more surface stabilizers adhered to the surface of the compound. The chemical compound incorporated in the resultant nanoparticulate composition exhibits increased chemical stability as compared to prior art formulations of the chemical compound.

### BACKGROUND OF THE INVENTION

Nanoparticulate compositions, first described in U.S. Pat. No. 5,145,684 (“the '684 patent”), are particles consisting of a poorly soluble therapeutic or diagnostic agent having adsorbed onto the surface thereof a non-crosslinked surface stabilizer.

A. Summary of Instability and/or Degradation of Chemical Compounds

Chemical compounds, whether in solid, liquid, gas, or semisolid products, decompose or degrade at various rates. Such decomposition or degradation may be due to hydrolysis, oxidation, isomerization, epimerization, or photolysis. The rate of degradation or decomposition varies considerably depending on the structural, physical, and chemical nature of the compound. The rate of decomposition is also often significantly affected by numerous environmental factors, including temperature, light, radiation, enzyme or other catalysts, pH and ionic strength of the solution, solvent type, and buffer species.

Chemical instability due to degradation or decomposition is highly undesirable for several reasons. For example, when a chemical compound is a pharmaceutical agent, degradation decreases its efficiency and shortens its effective shelf life. Moreover, the decrease in the content of the active ingredient in a pharmaceutical preparation renders the calculation of an effective dosage unpredictable and difficult. Furthermore, degraded chemical agent may have highly undesirable or even severely toxic side effects.

Because chemical stability is a critical aspect in the design and manufacture, as well as regulatory review and approval, of pharmaceutical compositions and dosage forms, in recent years extensive and systematic studies have been conducted on the mechanisms and kinetics of decomposition of pharmaceutical agents. For a brief review, see Alfred Martin, Physical Pharmacy: Physical Chemical Principles in the Pharmaceutical Sciences, 4^{th} Edition, pp. 305-312 (Lee & Febiger, Philadelphia, 1993).

### B. Prior Methods for Increasing the Stability of a Chemical Compound

1. Alteration of Environmental Parameters

Various methods have been devised to achieve improved chemical stability of a compound, including alteration of environmental parameters, such as buffer type, pH, storage temperature, and elimination of catalytic ions or ions necessary for enzyme activity using chelating agents.

2. Conversion of the Chemical Compound to a More Stable Prodrug

Other methods include converting the drug into a more stable prodrug which, under physiological conditions, is processed to become a biologically active form of the compound.

3. Novel Dosage Forms for Increasing the Chemical Stability of an Administered Agent

a. Liposomes or Particulate Polymeric Carriers

Another method for improving the chemical stability of pharmaceutical agents employs novel dosage form designs. Dosage form designs that improve the chemical stability of a drug include loading drugs into liposomes or polymers, e.g., during emulsion polymerization. However, such techniques have problems and limitations. For example, a lipid soluble drug is often required to prepare a suitable liposome. Further, unacceptably large amounts of the liposome or polymer may be required to prepare unit drug doses. Further still, techniques for preparing such pharmaceutical compositions tend to be complex. Finally, removal of contaminants at the end of the emulsion polymerization manufacturing process, such as potentially toxic unreacted monomer or initiator, can be difficult and expensive.

b. Monolithic and Reservoir Devices

Another example of a dosage form that can be used to increase the stability of an administered agent is a monolithic device, which is a rate-controlling polymer matrix throughout which a drug is dissolved or dispersed. Yet another example of such a dosage form is a reservoir device, which is a shell-like dosage form having a drug contained within a rate-controlling membrane.

An exemplary reservoir dosage form is described in U.S. Pat. No. 4,725,442, which refers to water insoluble drug materials solubilized in an organic liquid and incorporated in microcapsules of phospholipids. One disadvantage of this dosage form is the toxic effects of the solubilizing organic liquids. Other methods of forming reservoir dosage forms of pharmaceutical drug microcapsules include micronizing a slightly-soluble drug by high-speed stirring or impact comminution of a mixture of the drug and a sugar or sugar alcohol together with suitable excipients or diluents. See e.g. EP 411,629A. One disadvantage of this method is that the resultant drug particles are larger than those obtained with milling. Yet another method of forming a reservoir dosage form is directed to polymerization of a monomer in the presence of an active drug material and a surfactant to produce small-particle microencapsulation (International Journal of Pharmaceutics, 52:101-108 (1989)). This process, however, produces compositions containing contaminants, such as toxic monomers, which are difficult to remove. Complete removal of such monomers can be expensive, particularly when conducted on a manufacturing scale. A reservoir dosage form can also be formed by co-dispersion of a drug or a pharmaceutical agent in water with droplets of a carbohydrate polymer (see e.g. U.S. Pat. No. 4,713,249 and WO 84/00294). The major disadvantage of this procedure is that in many cases, a solubilizing organic co-solvent is required for the encapsulation procedure. Removal of traces of such harmful co-solvents can result in an expensive manufacturing process.

There is a need in the art for a method of stabilizing chemical compounds, which is efficient, cost-effective, and does not require the addition of potentially toxic solvents. The present invention satisfies this need.

### SUMMARY OF THE INVENTION

The present invention is directed to the discovery that chemical compounds, when formulated into nanoparticulate compositions, exhibit increased chemical stability. The increased stability can be evident, for example, following prolonged storage periods, exposure to elevated temperatures, or exposure to a non-physiological pH level.

One aspect of the invention is directed to a process for stabilizing chemical compounds, particularly pharmaceutical agents, comprising formulating a chemical compound into a nanoparticulate composition. The nanoparticulate composition comprises a poorly soluble crystalline or amorphous chemical compound, such as a drug particle, and one or more non-crosslinked surface stabilizers adsorbed on to the surface of the drug particle. The nanoparticulate compositions have an effective average particle size of less than about two microns.

The present invention is further directed to a process for stabilizing rapamycin, comprising forming a nanoparticulate formulation of rapamycin having one or more non-crosslinked surface stabilizers adsorbed on to the surface of the drug. The resultant nanoparticulate rapamycin composition exhibits dramatically superior stability, even following prolonged storage periods or exposure to elevated temperatures. The pharmaceutical composition preferably comprises a pharmaceutically acceptable carrier, as well as any desired excipients.

Yet another aspect of the invention encompasses a process for stabilizing paclitaxel, comprising forming a nanoparticulate formulation of paclitaxel having one or more non-crosslinked surface stabilizers adsorbed on to the surface of the drug. The resultant nanoparticulate paclitaxel composition exhibits dramatically superior stability even following prolonged storage periods, exposure to elevated temperature, or exposure to basic pH levels. The pharmaceutical composition preferably comprises a pharmaceutically acceptable carrier, as well as any desired excipients.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE FIGURE

FIG. 1: Shows the effect of 0.005 N NaOH (a basic pH level) on the rate of degradation of paclitaxel and on the rate of degradation of a nanoparticulate formulation of paclitaxel.

### DETAILED DESCRIPTION OF THE INVENTION

The claimed invention is directed to a method of chemically stabilizing a poorly water-soluble active agent, which is unstable under one or more environmental conditions, by formulating the active agent into a nanoparticulate composition. Such environmental conditions include, but are not limited to, exposure to water, unfavorable pH levels, repeated cycles of freezing and thawing, oxidizing agents or free radicals, or light.

The present invention is directed to a method for stabilizing chemical compounds, particularly pharmaceutical agents, comprising formulating a chemical compound into a nanoparticulate composition. The method according to the present invention enables chemical compounds to be stored for a prolonged period of time, and/or exposed to conditions which otherwise cause the chemical compound to degrade, such as exposure to elevated temperatures, water or other solvent molecules, or non-physiological pH levels.

A. Chemical Compounds Formulated into Nanoparticulate Compositions Exhibit Increased Stability of the Component Chemical Compound

It has been surprisingly discovered that a component chemical compound of a nanoparticulate composition exhibits superior stability as compared to the prior art chemical compound. Chemical instability due to degradation is usually a result of hydrolysis, oxidation, isomerization, epimerization, or photolysis. Apart from the structural, physical, and chemical nature of the compound, the rate of degradation is often determined by numerous environmental factors, including temperature, light, radiation, enzyme or other catalysts, pH and ionic strength of the solution, solvent type, or buffer species.

While not intending to be bound by theory, one possibility is that the molecules of the surface stabilizer shield the chemical compound, thereby protecting potentially labile chemical groups of the chemical compound from the potentially hostile environment. Another possibility is that for a crystalline drug particle, the crystalline structure in a nanoparticulate sized formulation results in greater drug stability.

For example, rapamycin is rapidly degraded when exposed to an aqueous environment. The main degradation scheme of rapamycin is the cleavage of the macrocyclic lactone ring by the hydrolysis of an ester bond to form a secoacid (SECO). The secoacid undergoes further dehydration and isomerization to form diketomorpholine analogs.

However, as described in the examples below, when rapamycin is formulated in a nanoparticulate composition, minimal or no rapamycin degradation is observed, even following prolonged exposure to an aqueous medium.

Another example of a drug that is unstable under certain environmental conditions, but which is stable in a nanoparticulate formulation under those same environmental conditions, is paclitaxel. Upon exposure to a basic pH (i.e., a pH of about 9), paclitaxel rapidly degrades. Ringel et al., J. Pharmac. Exp. Ther., 242:692-698 (1987). However, when paclitaxel is formulated into a nanoparticulate composition, minimal or no paclitaxel degradation is observed, even when the composition is exposed to a basic pH.

The process of increasing the stability of a chemical compound by formulating the compound into a nanoparticulate composition is broadly applicable to a wide range of drugs and active agents that are unstable and are poorly soluble under particular environmental conditions. Moreover, the process is also applicable to stabilization of a chemical compound under a broad range of environmental conditions which cause or aggravate chemical degradation, such as exposure to water (which can cause hydrolysis), unfavorable pH conditions, exposure to repeated freezing and thawing, exposure to oxidizing agents or other types of free radicals, or radiation causing photolysis.

### B. Methods of Preparing Nanoparticulate Compositions

1. Active Agent and Surface Stabilizer Components

The method of stabilizing a chemical compound according to the present invention comprises formulating the chemical compound into a nanoparticulate formulation. The nanoparticulate formulation comprises a drug and one or more surface stabilizers adsorbed to the surface of the drug.

a. Drug Particles

The nanoparticles of the invention comprise a therapeutic or diagnostic agent, collectively referred to as a “drug particle,” having one or more labile groups or exhibiting chemical instability when exposed to certain environmental conditions, such as elevated temperature, water or organic solvents, or non-physiological pH levels. A therapeutic agent can be a pharmaceutical, including biologics such as proteins and peptides, and a diagnostic agent is typically a contrast agent, such as an x-ray contrast agent, or any other type of diagnostic material. The drug particle exists as a discrete, crystalline phase or as an amorphous phase. The crystalline phase differs from a non-crystalline or amorphous phase which results from precipitation techniques, such as those described in EP Patent No. 275,796.

The invention can be practiced with a wide variety of drugs. The drug is preferably present in an essentially pure form, is poorly soluble, and is dispersible in at least one liquid medium. By “poorly soluble” it is meant that the drug has a solubility in the liquid dispersion medium of less than about 10 mg/mL, and preferably of less than about 1 mg/mL.

The drug can be selected from a variety of known classes of drugs, including, for example, proteins, peptides, nutriceuticals, anti-obesity agents, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immuriological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines.

A description of these classes of drugs and a listing of species within each class can be found in Martindale, The Extra Pharmacopoeia, Twenty-ninth Edition (The Pharmaceutical Press, London, 1989), specifically incorporated by reference. The drugs are commercially available and/or can be prepared by techniques known in the art.

b. Surface Stabilizers

Individually adsorbed molecules of the surface stabilizer are essentially free of intermolecular crosslinkages. Suitable surface stabilizers, which do not chemically interact with the drug particles, can preferably be selected from known organic and inorganic pharmaceutical excipients. Useful surface stabilizers include various polymers, low molecular weight oligomers, natural products, and surfactants. Preferred surface stabilizers include nonionic and ionic surfactants. Two or more surface auxiliary stabilizers can be used in combination. Representative examples of surface stabilizers include cetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Specialty Chemicals)); polyethylene glycols (e.g., Carbowaxs 3350® and 1450®, and Carbopol 934® (Union Carbide)), dodecyl trimethyl ammonium bromide, polyoxyethylene stearates, colloidal silicon dioxide, phosphates, sodium dodecylsulfate, carboxymethylcellulose calcium, hydroxypropyl celluloses (e.g., HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); a charged phospholipid such as dimyristoyl phophatidyl glycerol, dioctylsulfosuccinate (DOSS); Tetronic 1508® (T-1508) (BASF Wyandotte Corporation), dialkylesters of sodium sulfosuccinic acid (e.g., Aerosol OT®, which is a dioctyl ester of sodium sulfosuccinic acid (American Cyanamid)); Duponol P®, which is a sodium lauryl sulfate (DuPont); Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-1OG® or Surfactant 10-G®(Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂0H)₂ (Eastman Kodak Co.), and the like. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference. The surface stabilizers are commercially available and/or can be prepared by techniques known in the art.

c. Nanoparticulate Drug/Surface Stabilizer Particle Size

The compositions of the invention contain nanoparticles which have an effective average particle size of less than about 2 microns, less than about 1 micron, less than about 600 nm, less than about 500 nm, less than about 400 nm, less than about 300 nm, less than about 200 nm, less than about 100 nm, or less than about 50 nm, as measured by light-scattering methods, microscopy, or other appropriate methods. By “an effective average particle size of “less than about 2 microns,” it is meant that at least 50% of the drug particles have a weight average particle size of less than about 2 microns when measured by light scattering techniques, microscopy, or other appropriate methods. Preferably, at least 70% of the drug particles have an average particle size of less than about 2 microns, more preferably at least 90% of the drug particles have an average particle size of less than about 2 microns, and even more preferably at least about 95% of the particles have a weight average particle size of less than about 2 microns.

d. Concentration of Nanoparticulate Drug and Surface Stabilizer

The relative amount of drug and one or more surface stabilizers can vary widely. The optimal amount of the one or more surface stabilizers can depend, for example, upon the particular active agent selected, the hydrophilic lipophilic balance (HLB), melting point, and water solubility of the surface stabilizer, and the surface tension of water solutions of the surface stabilizer, etc.

The concentration of the one or more surface stabilizers can vary from about 0.1 to about 90%, and preferably is from about 1 to about 75%, more preferably from about 10 to about 60%, and most preferably from about 10 to about 30% by weight based on the total combined weight of the drug substance and surface stabilizer.

The concentration of the drug can vary from about 99.9% to about 10%, and preferably is from about 99% to about 25%, more preferably from about 90% to about 40%, and most preferably from about 90% to about 70% by weight based on the total combined weight of the drug substance and surface stabilizer.

2. Methods of Making Nanoparticulate Formulations

The nanoparticulate drug compositions can be made by, for example, milling or precipitation. Exemplary methods of making nanoparticulate compositions are described in U.S. Pat. No. 5,145,684.

Milling of aqueous drug to obtain a nanoparticulate dispersion comprises dispersing drug particles in a liquid dispersion medium, followed by applying mechanical means in the presence of grinding media to reduce the particle size of the drug to the desired effective average particle size of less than about 2 microns, less than about 1 micron, less than about 600 nm, less than about 500 nm, less than about 400 nm, less than about 300 nm, less than about 200 nm, less than about 100 nm, or less than about 50 nm. The particles can be reduced in size in the presence of one or more surface stabilizers. Alternatively, the particles can be contacted with one or more surface stabilizers after attrition. Other compounds, such as a diluent, can be added to the drug/surface stabilizer composition during the size reduction process. Dispersions can be manufactured continuously or in a batch mode. The resultant nanoparticulate drug dispersion can be utilized in all dosage formulations, including, for example, solid, liquid, aerosol, and nasal.

### C. Methods of Using Nanoparticulate Drug Formulations

The nanoparticulate compositions of the present invention can be administered to humans and animals either orally, rectally, parenterally (intravenous, intramuscular, or subcutaneous), intracisternally, intravaginally, intraperitoneally, locally (powders, ointments or drops), or as a buccal or nasal spray.

Compositions suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil), and injectable organic esters such as ethyl oleate.

Proper fluidity can be maintained, for example, by the use of a coating such as lecithin, by the maintenance of the required particle size in the case of dispersions, and by the use of surfactants. The nanoparticulate compositions may also contain adjuvants, such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include capsules, tablets, pills, powders, and granules. In such solid dosage forms, the active compound is admixed with at least one of the following: (a) one or more inert excipients (or carrier), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the active compounds, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Actual dosage levels of active ingredients in the nanoparticulate compositions of the invention may be varied to obtain an amount of active ingredient that is effective to obtain a desired therapeutic response for a particular composition and method of administration. The selected dosage level therefore depends upon the desired therapeutic effect, on the route of administration, on the desired duration of treatment, and other factors.

The total daily dose of the compounds of this invention administered to a host in single or divided dose may be in amounts of, for example, from about 1 nanomole to about 5 micromoles per kilogram of body weight. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

The following examples are given to illustrate the present invention. It should be understood, however, that the invention is not to be limited to the specific conditions or details described in these examples. Throughout the specification, any an all references to publicly available documents are specifically incorporated by reference.

### Example 1

The purpose of this example was to determine the effect on the stability of paclitaxel of formulating the drug into a nanoparticulate composition.

Paclitaxel is a naturally occurring diterpenoid which has demonstrated great potential as an anti-cancer drug. Paclitaxel can be isolated from the bark of the western yew, Taxus brevifolia, and is also found in several other yew species such as T. baccata and T. cuspidata. Upon exposure to a basic pH (i.e., a pH of about 9), the drug rapidly degrades. Ringel et al., J. Pharmac. Exp. Ther., 242:692-698 (1987).

Two formulations of paclitaxel were prepared: a solubilized formulation of paclitaxel and a nanoparticulate formulation of paclitaxel. The degradation of paclitaxel for both formulations was then compared. For Formulation I, paclitaxel (Biolyse; Quebec, Canada) was solubilized in 1% methanol and 99% H₂O to make a 2% paclitaxel solution. Formulation II was prepared by milling the 2% paclitaxel solution with 1% Plurionic F108™ (BASF) in a 0.5 oz amber bottle containing 7.5 ml 0.5 mm Yttria-doped Zirconia media on a U.S. Stoneware Roller Mill for 72 hours. The resultant milled composition had an effective average particle size of about 220 nm, as measured by a Coulter Counter (Coulter Electronics Inc.).

Both solubilized paclitaxel (Formulation I) and nanoparticulate paclitaxel (Formulation II) were incubated with 0.005 N NaOH solution (a basic solution). At the end of the incubation period, base degradation of paclitaxel was stopped by adding to the incubation solution 1/100 its volume of 1N HCl. The recovery of paclitaxel was then measured at various time periods by HPLC.

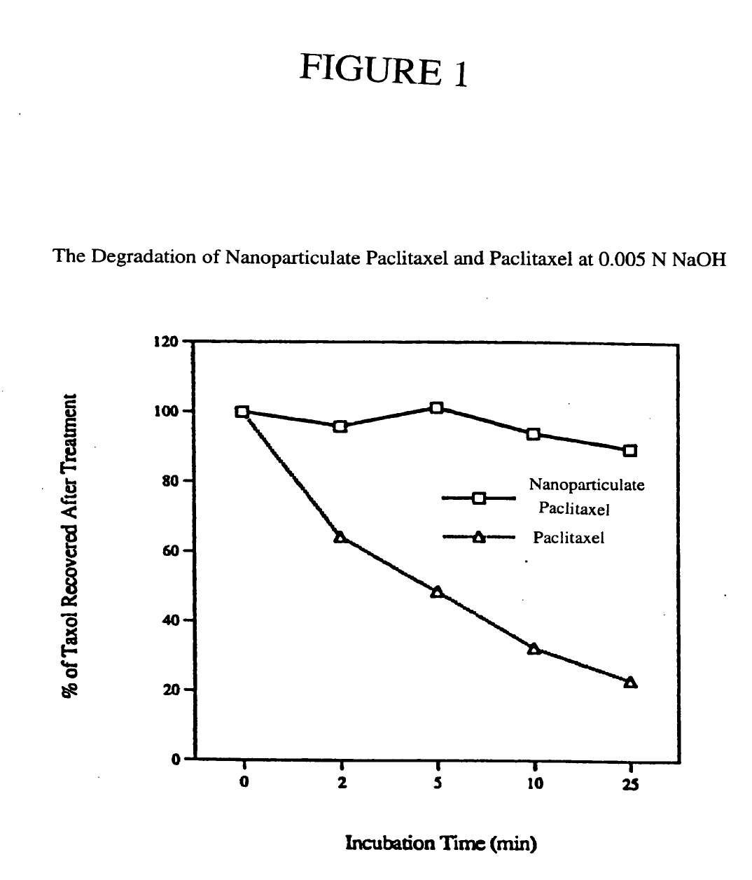

As shown in FIG. 1, solubilized paclitaxel rapidly degraded when exposed to basic conditions, as only about 20% of the paclitaxel was recoverable after a 20 minute incubation period. In contrast, nanoparticulate paclitaxel was essentially stable under basic conditions, as more than 90% of the drug was recoverable after the same incubation period.

### Example 2

The purpose of this example was to determine the effect on the stability of rapamycin of formulating the drug into a nanoparticulate composition.

Rapamycin is useful as an immunosuppressant and as an antifungal antibiotic, and its use is described in, for example, U.S. Pat. Nos. 3,929,992, 3,993,749, and 4,316,885, and in Belgian Pat. No. 877,700. The compound, which is only slightly soluble in water, i.e., 20 micrograms per mL, rapidly hydrolyzes when exposed to water. Because rapamycin is highly unstable when exposed to an aqueous medium, special injectable formulations have been developed for administration to patients, such as those described in European Patent No. EP 041,795. Such formulations are often undesirable, as frequently the non-aqueous solubilizing agent exhibits toxic side effects.

Two different formulations of rapamycin were prepared and then exposed to different environmental conditions. The degradation of rapamycin for each of the formulations was then compared. The two formulations were prepared as follows:
(1) Formulation I, a mixture of 5% rapamycin and 2.5% Plurionic F68™ (BASF) in an aqueous medium; and
(2) Formulation II, a mixture of 5% rapamycin and 1.25% Plurionic F108™ (BASF) in an aqueous medium.

Each of the two formulations was milled for 72 hours in a 0.5 ounce bottle containing 0.4 mm Yttria beads (Performance Ceramics Media) on a U.S. Stoneware Mill. Particle sizes of the resultant nanoparticulate compositions were measured by a Coulter Counter (Model No. N4MD). Following milling, Formulations I and II had effective average particle sizes of 162 nm and 171 nm, respectively.

The samples were then diluted to about 2% rapamycin with Water For Injection (WFI), bottled, and then either stored at room temperature or frozen upon completion of milling and then thawed and stored at room temperature. After ten days of storage at room temperature, Formulations I and II had effective average particle sizes of 194 nm and 199 nm, respectively.

The strength of the rapamycin in the formulations was measured by HPLC, the results of which are shown below in Table I.

TABLE I
Stability of Nanoparticulate Rapamycin under Different
Storage Conditions

| | | | | Ending | |
|---|---|---|---|---|---|
| | | | | Strength/ | |
| Sam- | | Storage | Storage | Starting | |
| ple | Description | Conditions | Time | Strength | SECO %* |
| 1 | Formulation I | RT | 2 days | 97% | <detection limit |
| 2 | Formulation II | RT | 2 days | 99% | <detection limit |
| 3 | Formulation III | RT | 2 days | 96% | <detection limit |
| 7 | Formulation I | Frozen/ | 2 days | 95% | <detection limit |
| | | thawed | | | |
| 8 | Formulation II | Frozen/ | 2 days | 98% | <detection limit |
| | | thawed | | | |
| 9 | Formulation III | Frozen/ | 2 days | 97% | <detection limit |
| | | thawed | | | |
| 1 | Formulation I | RT | 3 wks | 95% | <detection limit |
| 2 | Formulation II | RT | 3 wks | 98% | <detection limit |
| 3 | Formulation III | RT | 3 wks | 98% | <detection limit |

*SECO, or secoacid, is the primary degradation product of rapamycin. The detection limit is 0.2%.

The results show that the nanoparticulate rapamycin formulation exhibited minimal degradation of rapamycin following prolonged storage periods or exposure to the environmental conditions of freezing and thawing.

### Example 3

The purpose of this example was to determine the effect of rapamycin concentration on the chemical stability of rapamycin in a nanoparticulate formulation following autoclaving.

Three rapamycin formulations were prepared by milling the following three slurries in a 250 ml Pyrex™ bottle containing 125 ml 0.4 mm Yttria-doped Zirconia media for 72 hours on a U.S. Stoneware roller mill:

(a) 5% rapamycin/1.25% Plurionic F68™

(b) 5% rapamycin/2.5% Plurionic F68™

(c) 5% rapamycin/5% Plurionic F68™

Each of the three dispersions was then diluted with water to prepare formulations having rapamycin concentrations of 4.4%, 2.2%, 1.1% and 0.5% as follows:
(1) Formulation 1: a mixture of 4.4% rapamycin and, prior to dilution, 1.25% Plurionic F68™ in an aqueous medium;
(2) Formulation 2: a mixture of 4.4% rapamycin and, prior to dilution, 2.5% Plurionic F68™ in an aqueous medium;
(3) Formulation 3: a mixture of 4.4% rapamycin and, prior to dilution, 5% Plurionic F68™ in an aqueous medium;
(4) Formulation 4: a mixture of 2.2% rapamycin and, prior to dilution, 1.25% Plurionic F68™ in an aqueous medium;
(5) Formulation 5: a mixture of 2.2% rapamycin and, prior to dilution, 2.5% Plurionic F68™ in an aqueous medium;
(6) Formulation 6: a mixture of 2.2% rapamycin and, prior to dilution, 5% Plurionic F68™ in an aqueous medium;
(7) Formulation 7: a mixture of 1.1% rapamycin and, prior to dilution, 1.25% Plurionic F68™ in an aqueous medium;
(8) Formulation 8: a mixture of 1.1% rapamycin and, prior to dilution, 2.5% Plurionic F68™ in an aqueous medium;
(9) Formulation 9: a mixture of 1.1% rapamycin and, prior to dilution, 5% Plurionic F68™ in an aqueous medium;
(10) Formulation 10: a mixture of 0.55% rapamycin and, prior to dilution, 1.25% Plurionic F68™ in an aqueous medium;
(11) Formulation 11: a mixture of 0.55% rapamycin and, prior to dilution, 2.5% Plurionic F68™ in an aqueous medium; and
(12) Formulation 12: a mixture of 0.55% rapamycin and, prior to dilution, 5% Plurionic F68™ in an aqueous medium;

All twelve of the nanoparticulate formulations were autoclaved for 25 minutes at 121° C. The formulations were then stored at 4° C. for 61 days, followed by testing for rapamycin degradation. No degradation, as measured by the percent of the SECO degradation product, was detected for any of the formulations.

### Example 4

The purpose of this example was to determine the chemical stability of a nanoparticulate rapamycin formulation following a prolonged storage period at room temperature.

A mixture of 20% rapamycin and 10% Plurionic F68™ in an aqueous medium was milled with 0.4 mm YTZ media (Performance Ceramic Co.) on a U.S. Stoneware mill for 72 hours at room temperature. The final nanoparticulate composition had a mean particle size of between 180 to 230 nm, as measured by Coulter sizing.

After two weeks of storage at room temperature, no SECO degradation product was detected in any of the nanoparticulate preparations, indicating that there was minimal or no degradation of rapamycin in the stored nanoparticulate formulation samples.

### Example 5

The purpose of this example was to determine the effect of long term storage on the chemical stability of rapamycin in a nanoparticulate composition.

Three different nanoparticulate rapamycin formulations were prepared as follows: Formulation 1, having a rapamycin concentration of 182.8 mg/mL; Formulation 2, having a rapamycin concentration of 191.4 mg/mL; and Formulation 3, having a rapamycin concentration of 192.7 mg/mL.

The formulations were prepared by milling the following three slurries in a 0.5 oz amber bottle containing 7.5 ml 0.8 mm Yttria-doped Zirconia media for 72 hours on a U.S. Stoneware roller mill:

(1) 20% rapamycin/10% Plurionic F68

(2) 20% rapamycin/5% Plurionic F68

(3) 20% rapamycin/2.5% Plurionic F68

Following storage for two and half months, no SECO degradation product was detected in any of the samples. These results show that various dosage strengths of rapamycin can be used in nanoparticulate formulations without any impact on the increased chemical stability of the drug.

It will be apparent to those skilled in the art that various modifications and variations can be made in the methods and compositions of the present invention without departing from the spirit or scope of the invention. Thus, it is intended that the present invention cover the modifications and variations of this invention, provided they come within the scope of the appended claims and their equivalents.

1.-19. (canceled)

20. A chemically stable nanoparticulate paclitaxel composition comprising:
(a) paclitaxel particles having an effective average particle size of less than about 2 microns; and
(b) at least one surface stabilizer adsorbed to the surface of the paclitaxel particles.

21. The composition of claim 20, wherein the paclitaxel particles are in a crystalline phase or in an amorphous phase.

22. The composition of claim 20, wherein the surface stabilizer is non-crosslinked.

23. The composition of claim 20, wherein the surface stabilizer is selected from the group consisting of nonionic surfactants and ionic surfactants.

24. The composition of claim 20, comprising two or more surface stabilizers.

25. The composition of claim 20, wherein the surface stabilizer is selected from the group consisting of cetyl pyridinium chloride, gelatin, casein, lecithin, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol, polyvinylpyrrolidone, tyloxapol, poloxamers, block copolymers of ethylene oxide and propylene oxide, poloxamines, a charged phospholipid, dimyristoyl phophatidyl glycerol, dioctylsulfosuccinate (DOSS), Tetronic 1508®, dialkylesters of sodium sulfosuccinic acid, dioctyl ester of sodium sulfosuccinic acid, sodium lauryl sulfate, an alkyl aryl polyether sulfonate, a mixture of sucrose stearate and sucrose distearate, p-isononylphenoxypoly-(glycidol), Crodestas SL-40®, and C₁₈H₃₇CH₂(CON(CH₃)—CH₂(CHOH)₄(CH20H)₂.

26. The composition of claim 20, wherein the effective average particle size of the paclitaxel particles is selected from the group consisting of less than about 1 micron, less than about 600 nm, less than about 500 nm, less than about 400 nm, less than about 300 nm, less than about 200 nm, less than about 100 nm, and less than about 50 nm.

27. The composition of claim 20, wherein:
(a) at least 70% of the paclitaxel particles have a size of less then 2 microns;
(b) at least 90% of the paclitaxel particles have a size of less then 2 microns; or
(c) at least 95% of the paclitaxel particles have a size of less then 2 microns.

28. The composition of claim 20, wherein the at least one surface stabilizer is present in an amount of between about 0.1% to about 90%, between about 1% to about 75%, between about 10% to about 60%, and between about 10% to about 30%, by weight, based on the total combined weight of paclitaxel and surface stabilizer.

29. The composition of claim 20, wherein paclitaxel is present in an amount of between about 99.9% to about 10%, between about 99% to about 25%, between about 90% to about 40%, and between about 90% to about 70%, by weight, based on the total combined weight of paclitaxel and surface stabilizer.

30. The composition of claim 20, wherein the surface stabilizer is a poloxamer.

31. The composition of claim 20, further comprising at least one pharmaceutically acceptable carrier.

32. The composition of claim 20, wherein the composition is chemically stable upon exposure to a basic pH.

33. The composition of claim 20, wherein the composition is chemically stable under conditions which would cause a non-nanoparticulate dosage form of paclitaxel to chemically degrade as a result of hydrolysis, oxidation, isomerization, epimerization, or photolysis.

34. The composition of claim 33, wherein the conditions which would result in chemical degradation of a non-nanoparticulate dosage form of paclitaxel are selected from the group consisting of exposure to water, exposure to repeated cycles of freezing and thawing, oxidizing agents, free radicals, light, and unfavorable pH conditions.

35. The composition of claim 20 formulated into a dosage form for administration orally, rectally, parenterally, intracisternally, intravaginally, intraperitoneally, locally, as a buccal spray, or as a nasal spray.

36. The composition of claim 20 formulated into a dosage form selected from the group consisting of solid dosage form or oral administration, liquid dosage form for oral administration, or an injectable dosage form.

37. A method for chemically stabilizing paclitaxel under conditions selected from prolonged storage period, hydrolysis, oxidation, isomerization, epimerization, photolysis, repeated cycles of freezing and thawing, exposure to elevated temperature and exposure to basic pH levels, comprising forming a nanoparticulate paclitaxel composition comprising paclitaxel particles having an effective average particle size of less than about 2 microns, and at least one surface stabilizer adsorbed to the surface of the paclitaxel particles.

38. The method of claim 37, wherein the paclitaxel particles are in a crystalline phase or in an amorphous phase.

39. The method of claim 37, wherein the surface stabilizer is non-crosslinked.

40. The method of claim 37, wherein the surface stabilizer is selected from the group consisting of nonionic surfactants and ionic surfactants.

41. The method of claim 37, comprising two or more surface stabilizers.

42. The method of claim 37, wherein the surface stabilizer is selected from the group consisting of cetyl pyridinium chloride, gelatin, casein, lecithin, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol, polyvinylpyrrolidone, tyloxapol, poloxamers, block copolymers of ethylene oxide and propylene oxide, poloxamines, a charged phospholipid, dimyristoyl phophatidyl glycerol, dioctylsulfosuccinate (DOSS), Tetronic 1508®, dialkylesters of sodium sulfosuccinic acid, dioctyl ester of sodium sulfosuccinic acid, sodium lauryl sulfate, an alkyl aryl polyether sulfonate, a mixture of sucrose stearate and sucrose distearate, p-isononylphenoxypoly-(glycidol), Crodestas SL-40®, and C₁₈H₃₇CH₂(CON(CH₃)—CH₂(CHOH)₄(CH20H)₂.

43. The method of claim 37, wherein the effective average particle size of the paclitaxel particles is selected from the group consisting of less than about 1 micron, less than about 600 nm, less than about 500 nm, less than about 400 nm, less than about 300 nm, less than about 200 nm, less than about 100 nm, and less than about 50 nm.

44. The method of claim 37, wherein:
(a) at least 70% of the paclitaxel particles have a size of less then 2 microns;
(b) at least 90% of the paclitaxel particles have a size of less then 2 microns; or
(c) at least 95% of the paclitaxel particles have a size of less then 2 microns.

45. A method for making a chemically stable nanoparticulate paclitaxel composition comprising milling paclitaxel in the presence of a liquid dispersion medium to reduce the effective average particle size of paclitaxel to less than about 2 microns, wherein at least one surface stabilizer is added before, during, or after the milling.
